(19) United States
(12) Patent Application Publication
(10) Pub. No.: US 2013/0066246 A1
(43) Pub. Date: Mar. 14, 2013

(54) ORTHOPEDIC CAST WALKING SUPPORT

(71) Applicants: Kishore Tipirneni, Glendale, AZ (US); Todd Richard Christopherson, Glendale, AZ (US)

(72) Inventors: Kishore Tipirneni, Glendale, AZ (US); Todd Richard Christopherson, Glendale, AZ (US)

(73) Assignee: CASHOE L.L.C., Glendale, AZ (US)

(21) Appl. No.: 13/614,901

(22) Filed: Sep. 13, 2012

Related U.S. Application Data
(60) Provisional application No. 61/573,813, filed on Sep. 14, 2011.

(51) Int. Cl. A61F 5/00; B32B 38/10; B32B 37/12

(52) U.S. Cl. 602/12; 156/60; 156/250

(57) ABSTRACT

The present disclosure relates to a support apparatus for use on a leg, ankle, and foot cast. The support apparatus attaches to the bottom of the cast and has a first sole and a second sole. The first sole is removable from the second sole providing the cast with both an aggressive tread on the second sole and a moderate tread on the first sole.

### CROSS-REFERENCE TO RELATED APPLICATIONS

This application claims priority to U.S. provisional patent application Ser. No. 61/573,813, filed on Sep. 14, 2011, entitled “THE CHRISSY SOLE,” which is hereby incorporated by reference in its entirety for all references.

### FIELD OF DISCLOSURE

The present disclosure relates generally to an apparatus for supporting and allowing a patient to walk on a leg or ankle cast.

### BACKGROUND OF THE DISCLOSURE

Leg, ankle and foot casts are common tools for treating patients. The cast by itself (commonly made from fiber glass or plaster) is hard and has an irregular shape. Walking on a cast in its natural state (no boot or cast shoe) causes additional problems and challenges, such as the following: the cast has a tendency to retain dirt and other debris, the casting material breaks down with constant successive force applied, the shape of the cast shell is not sufficiently ergonomic for walking and the cast does not provide an adequately stable tracking surface relative to traction and stability. Furthermore, the industry standard means and methods for applying a leg, ankle and foot cast inherently has its own disadvantage. For example, it is difficult to cure a cast with weight on it. Thus, the patient's limb can, potentially be retained in a position that is not comfortable to walking because the cast was cured in a non-walking position. The result of this is that occasionally, a leg, ankle and foot cast needs to be removed and redone to be sure that the leg/ankle/foot are in an appropriate and comfortable position necessary for healing. Solutions to these and other cast support problems are desirable.

### SUMMARY OF THE DISCLOSURE

In accordance with various embodiments, the present disclosure includes an apparatus providing support for a cast. The apparatus comprises a first sole (e.g., upper sole) adhered to the bottom of the cast. The bottom of the first sole includes a gripping surface (e.g., a tread pattern). A second sole (e.g., lower sole) adheres to the bottom of the first sole. The second sole is removably adhered to the first sole using any means known in the art for adhering two objects. For example, the bottom of the first sole includes a plurality of holes suitable for receiving a plurality of studs. The second sole includes a plurality of studs on the top of the sole. The plurality of studs on the top of the second sole engages the plurality of holes on the bottom of the first sole adhering the two soles together. The plurality of studs include a circumferential rib around the stud more than half way up the stud measuring from the top surface of the second sole to the top of the stud. The disclosure contemplates the holes and/or studs to be fully or partially on the first sole and/or second sole.

The first sole includes a cushion between the upper and lower surfaces of the sole. In various embodiments, the first sole cushion may be thicker at the heel than the rest of the first sole. The first sole includes molded trim lines on the top surface enabling simplified alteration to fit a variety of cast sizes. The first sole may include grooves on the top surface and side walls which contact the cast. The grooves indicate locations to apply adhesive and improve adhesion to the cast. The second sole includes a gripping surface (e.g., a tread pattern) on the bottom of the second sole. The second sole tread pattern may be more aggressive than the first sole tread pattern. The second sole may include a tab on the back which extends beyond the contacting surfaces of the first sole and the second sole assisting in easy removal of the second sole.

In accordance with various embodiments, the present disclosure provides a method for constructing a cast support which may comprise wrapping a casting material around the lower extremity of a patient, applying an adhesive to the grooves located on the top surface and side walls of the first sole, compressing the upper surface and sidewalls of the first sole against the bottom and sides of the cast (e.g., prior to allowing the casting material to cure) and placing weight in the cast further compressing the first sole and the cast material (e.g., prior to allowing the casting material to cure). A second sole may be applied to the bottom of the first sole for outdoor use. The first sole and/or second sole may be cut along a line molded into the sole to adapt the first sole and/or the second sole to the size of the cast. The second sole may be removed from the bottom of the first sole for indoor use.

### BRIEF DESCRIPTION OF THE DRAWINGS

The drawings described herein are for illustration purposes only and are not intended to limit the scope of the present disclosure in any way. The present disclosure will become more fully understood from the detailed description and the accompanying drawings wherein:

FIG. 1a is a side view of a cast and a cast support apparatus, in accordance with various embodiments;

FIG. 1b is a cross-section side view of a cast and a cast support apparatus, in accordance with various embodiments;

FIG. 1c is a rear view of a cast and a cast support apparatus, in accordance with various embodiments;

FIG. 1d is a bottom view of a cast and a cast support apparatus, in accordance with various embodiments;

FIG. 2 is an isometric view of a first sole of a cast support apparatus, in accordance with various embodiments;

FIG. 3 is an isometric view of a second sole of a cast support apparatus, in accordance with various embodiments;

FIG. 4 is a side view of a cast support apparatus, in accordance with various embodiments;

FIG. 5 is a rear view of a cast support apparatus, in accordance with various embodiments;

FIG. 6 is a top view of a cast support apparatus, in accordance with various embodiments;

FIG. 7 is a bottom view of a first sole of a cast support apparatus, in accordance with various embodiments;

FIG. 8 is a side view of a second sole of a cast support apparatus, in accordance with various embodiments;

FIG. 9 is a bottom view of a second sole of a cast support apparatus, in accordance with various embodiments; and

FIG. 10 is a cast and cast apparatus assembly method, in accordance with various embodiments.

### DETAILED DESCRIPTION

The detailed description of exemplary embodiments herein makes reference to the accompanying drawings, which show exemplary embodiments by way of illustration and its best mode. While these exemplary embodiments are described in sufficient detail to enable those skilled in the art to practice the invention, it should be understood that other embodiments may be realized and that logical, chemical and mechanical changes may be made without departing from the spirit and scope of the invention. Thus, the detailed description herein is presented for purposes of illustration only and not of limitation. For example, the steps recited in any of the method or process descriptions may be executed in any order and are not necessarily limited to the order presented. Moreover, many of the functions or steps may be outsourced to or performed by one or more third parties. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments, the present disclosure provides a support apparatus for a cast on any person, animal, plant or object. The cast may be used on a patient's extremities, and in various embodiments, the lower extremities (e.g. leg or ankle). The support apparatus may provide support to the bottom of the cast to allow the cast to be converted to a weight bearing (e.g., walking) cast. For example, the support apparatus may comprise at least one sole. The sole may include at least a tread portion configured to prevent (or reduce) the ground from damaging either the cast or any other portion of the sole. The sole may also include a cushion portion which is configured to prevent (or reduce) damage to the cast and improve the comfort of walking in the cast. The cushion portion of the sole may be attached to the bottom of the cast. The tread portion of the sole may be attached to the bottom of the cushion. Providing both a cushion portion and a tread portion on the sole improves the traction, the stability, and/or the shock resistance (cushioning) of the cast. Furthermore, the sole may provide gait preservation allowing a patient to walk in a manner similar to one in which the patient would walk without the cast. With these improvements, a patient is more easily able to use the cast as a walking cast comfortably and for longer periods of time.

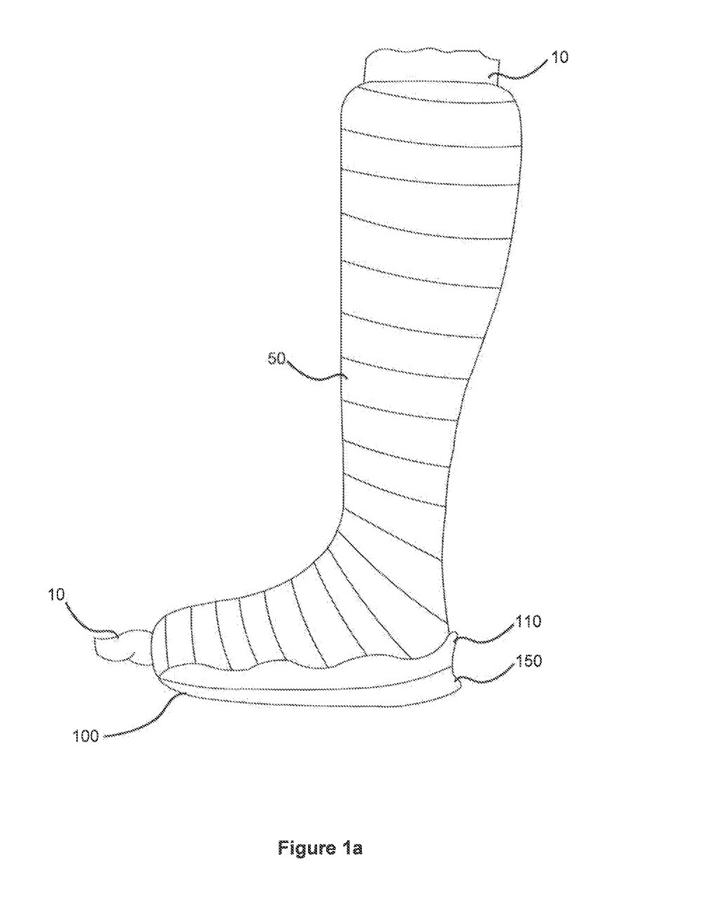

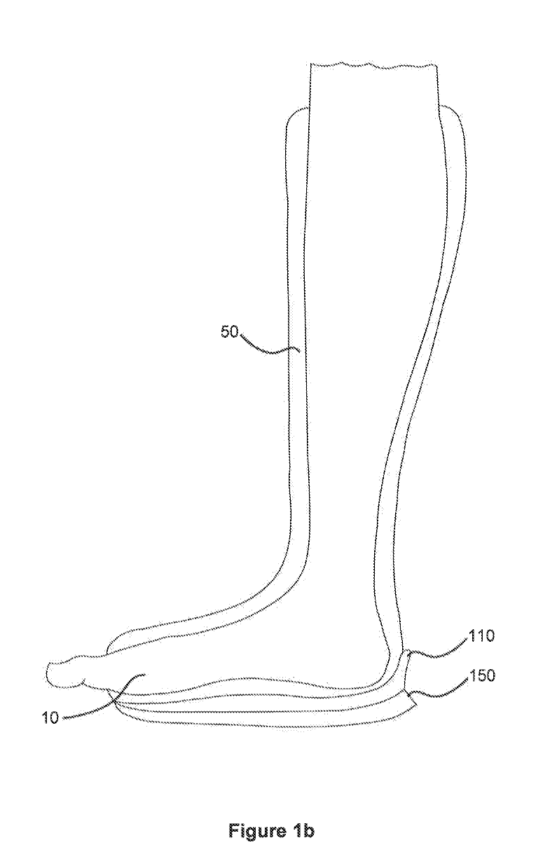

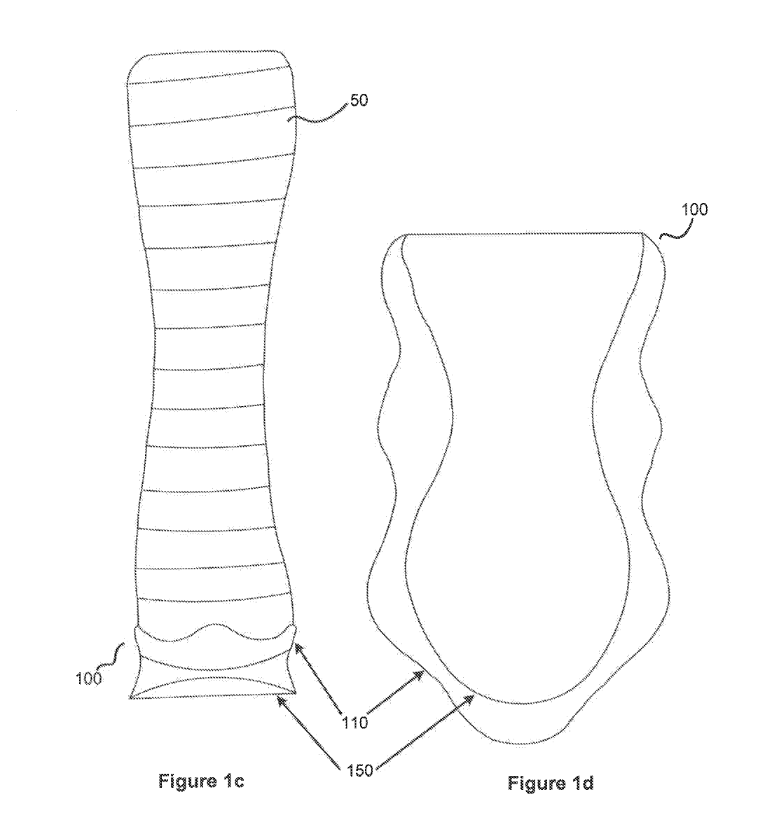

In various embodiments, and as illustrated in FIG. 1a, support apparatus 100 may comprise more than one sole. For example, support apparatus 100 may comprise a first sole 110 and a second sole 150. First sole 110 may be configured to attach to the bottom of a new or an existing cast 50 located on a patients leg/ankle 10. The second sole 150 may be configured to attach to the bottom of the first sole 110. Second sole 150 may be specifically configured to protect cast 50 and the cast support apparatus 100 against a rough or rugged walking surface. First sole 110 may be specifically configured to protect cast 50 and cast support apparatus 100 against a gentle walking surface and provide the comfort improvements discussed herein.

In various embodiments, the support apparatus may be permanently or temporarily attached to the bottom of the cast. For example, the first sole may be permanently attached to the bottom of the cast with the second sole removably attached to the bottom of the first sole. The first sole may be permanently attached to the new or existing leg/ankle cast through the use of an adhesive. Alternatively in another example, the first sole may be removably attached to the bottom of the cast with the second sole removably attached to the first sole. In still another example, the first sole may be removably attached to the bottom of the cast with the second sole permanently attached to the first sole.

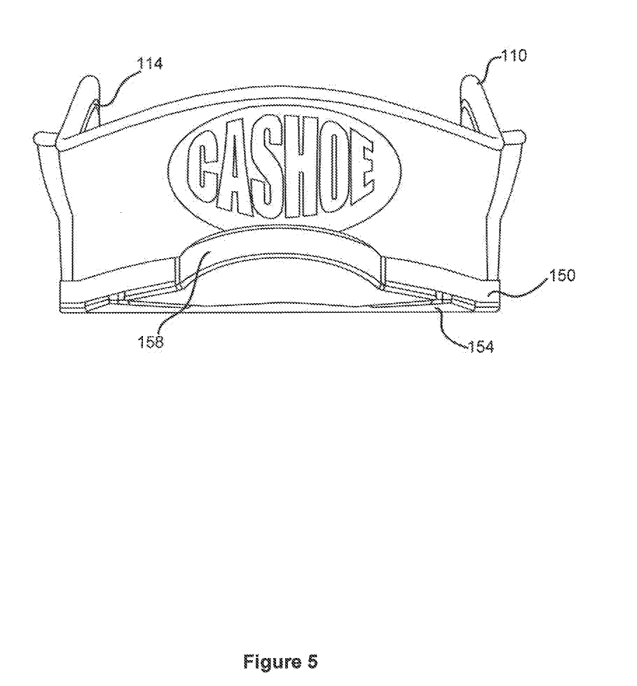

As illustrated in FIGS. 1-7, first sole 110 may be constructed as a one-piece unit. First sole 110 may be constructed from natural or synthetic, flexible and/or cushioning material. For example, the first sole may be constructed from a rubber, foam or plastic material. More specifically, a soft rubber may be used which provides a good cushion but may sacrifice durability.

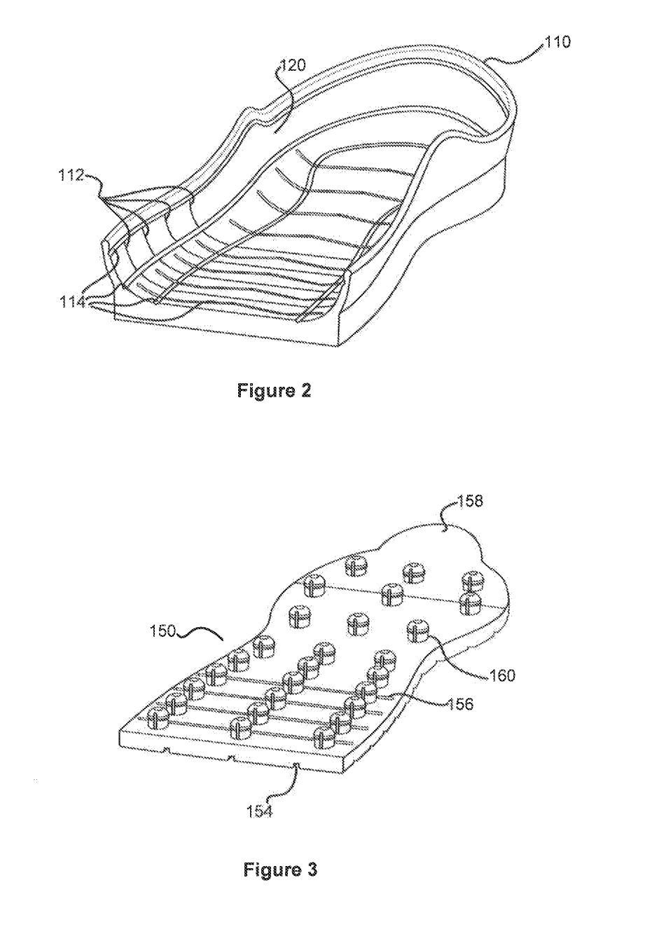

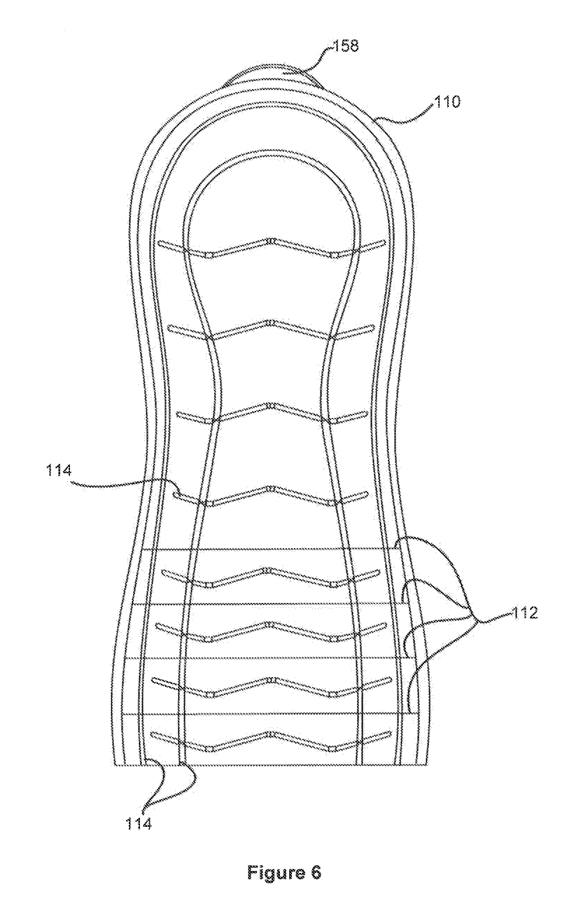

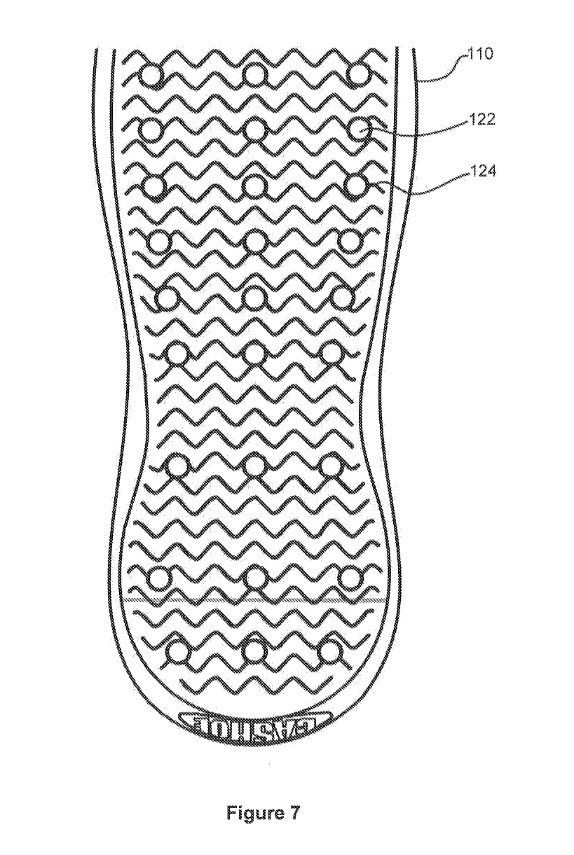

First sole 110 may have a top surface illustrated in FIGS. 2 and 6 and a bottom surface illustrated in FIG. 7, first sole 110 may also have side walls 120 that can adjust to the contours of differing cast shapes and reach around the side of cast 50 when attaching first sole 110 to cast 50. First sole 110 may comprise a flared-out shape. For example, first sole 110 may flare out from the sole's top surface (i.e. the surface that contacts the cast) to the bottom surface (i.e. the surface that would contact the ground or the second sole). This flared-out shape provides suitable surface contact area between the first sole and the cast, and increased contact area with the ground and/or second sole to provide better traction and stability.

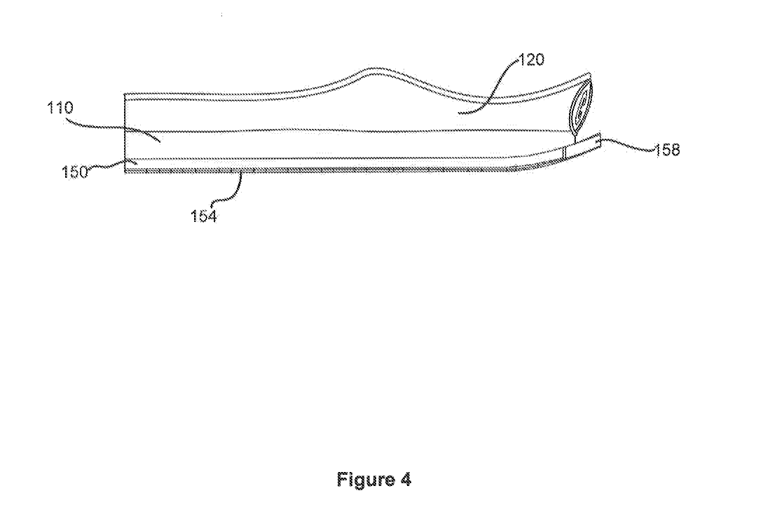

In various embodiments, the first sole may have a cushion that is thinner near the front (towards the toes) and proceeds to a thicker cushion in the back (near the heal). This change in the first sole cushion thickness from the front to the back provides a more ergonomic and comfortable sole for the cast, as well as additional cushioning (shock resistance) at the primary area of contact (heal strike) with the walking surface. In various embodiments as illustrated in FIG. 4, the back of the first sole may curve from the base up the rear of the first sole providing a contour beneficial to walking. This shape may enable a patient to easily roll from a heal strike onto the bottom of the sole. In various embodiments, the first sole may have a curved shape along the length of its bottom surface, when adhered to the cast, functioning as an ergonomic rocker bottom.

As illustrated in FIG. 7, the first sole may have a tread 124 on the bottom surface. In various embodiments, tread 124 does not interfere with adhesion to the second sole. For example, tread 124 may be one or more grooves cut into the cushion material but leaving a substantially flat bottom of the first sole such that the bottom of the first sole maintains good contact with a second sole when the two are attached. In accordance with various embodiments, an additional layer of durable flat tread may be permanently attached to the cushion to provide a ware surface on the bottom of the first sole. In these various examples, the cushioning, traction, and stability features of the cast support are not lost in response to removal of the second sole.

As illustrated in FIG. 7, the first sole may further comprise various attachment features for connecting the first sole to a second sole. For example, the first sole may include a plurality of holes 122 for receiving a plurality of mounting features which are a part of the second sole. Using holes 122 as the mounting feature on the first sole prevents or reduces the mounting feature from being degraded when walking on only the first sole. The attachment feature on the first sole is preferably a feature that does not come into contact with the ground when walking using only the first sole.

As illustrated in FIGS. 2 and 6, first sole 110 may include trim lines 112. Trim lines 112 may include lines or areas of the cushion that are thinner than others that are molded into the first sole. Trim lines 112 indicate lengths that the first sole may be cut to in order to fit casts of various sides. The trim lines also provide slightly thinner cushion area that is cut easier.

In various embodiments, the first sole may be attached to the cast with an adhesive. One of ordinary skill in the art will appreciate the variety of adhesives that may be used and can be selected from any of animal, plant, solvent synthetic monomer, and synthetic polymer glues. For example, for a quick, easy, safe and nontoxic application, the adhesive may be hot glue, double-backed foam tape, quick-set industrial liquid adhesives, or similar. In accordance with various embodiments, as illustrated in FIGS. 2 and 6, the first sole may include adhesive lines 114. Adhesive lines 114 may be lines on the top surface of the first sole. Adhesive lines 114 may be grooves molded into the upper surface that indicate specific locations that an adhesive should be applied to the surface in order to properly adhere the first sole to the cast. The first sole may improve stability and traction of the cast when using the cast as a walking cast. The combination of the first sole embodiments and improvements discussed herein taken singly (or in any combination) provides a more stable and safer condition for patients who need to utilize their cast in an active, weight-bearing manner.

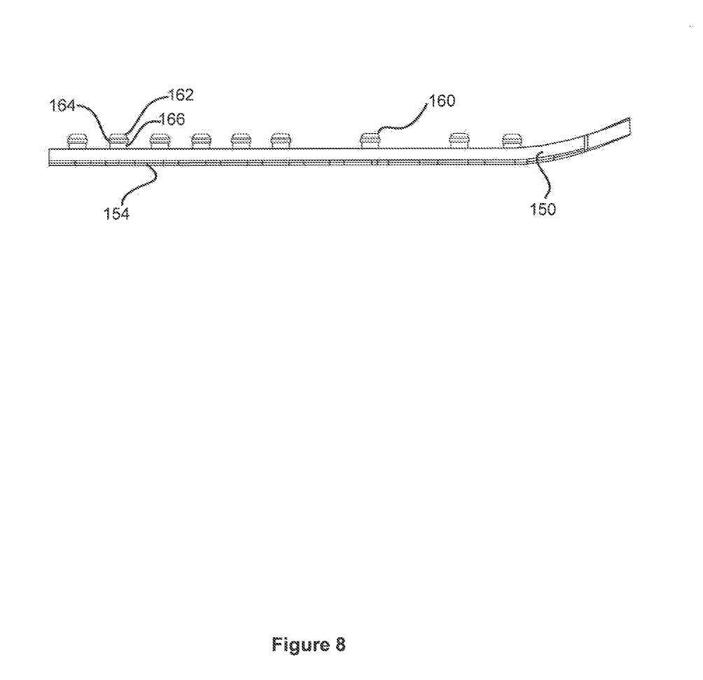

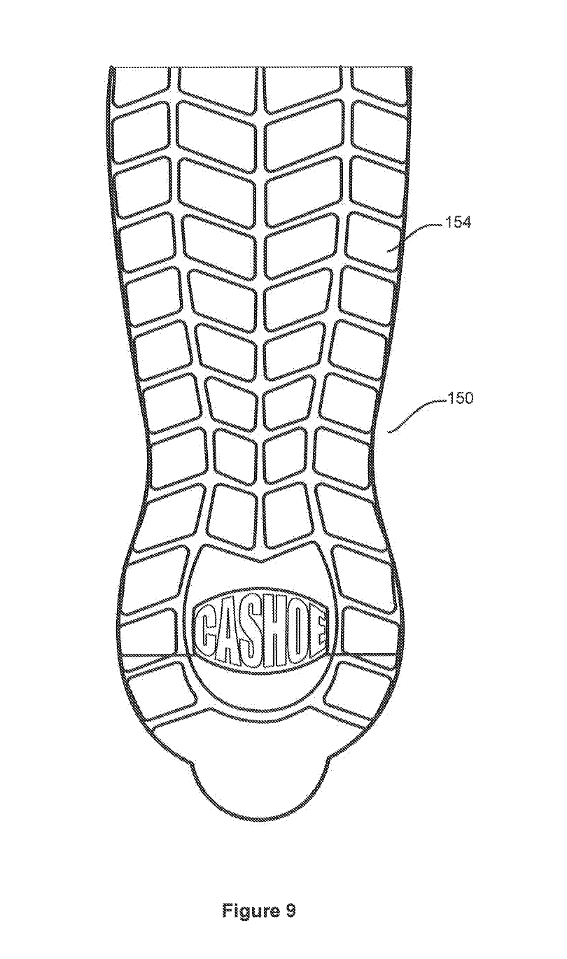

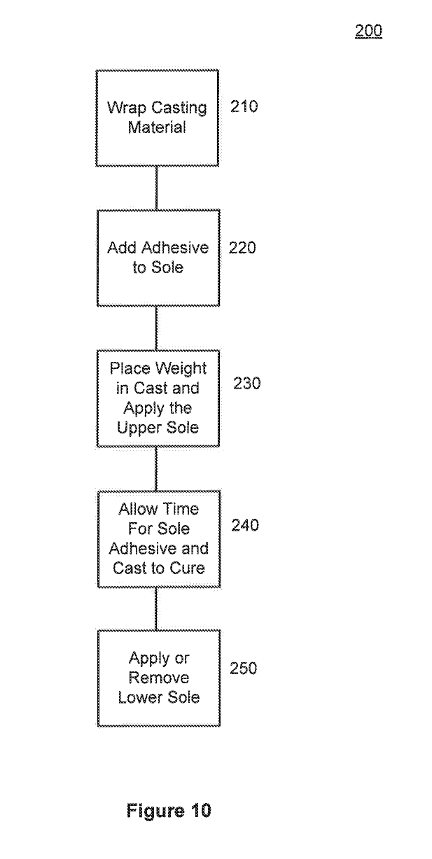

The benefits of first sole 110 may be further realized by incorporating use of a second sole 150 as discussed above. In various embodiments, as illustrated in FIGS. 3, 4, 5, 8 and 9, second sole 150 may be constructed as a one-piece unit. The second sole may be constructed from natural or synthetic material. For example, the second sole may be constructed from a harder rubber, foam or plastic material that promotes durability and good traction. For example, second sole 150 may have a very aggressive tread 154 with large lugs. Second sole 150 does not necessarily mate with another sole on its bottom like the first sole. As such second sole 150 may have a tread pattern that is more aggressive. In accordance with one embodiment, the second sole with its intrinsic properties (e.g. durable aggressive tread) is suitable for indoor or outdoor use. However; the second sole may be removed as discussed herein and the first sole with its intrinsic properties (e.g. less durable tread) may be suitable for indoor use.

As discussed herein, first sole 110 and second sole 150 may be attached to one another. While this attachment may be performed by any means necessary, it is desirable to have an attachment that does limit use of the first sole without the second sole. In various embodiments, the attachment may be accomplished by inserting posts into holes and using friction or interference to limit the two from being separated. Specifically, second sole 150 may include posts 160 which extend from the upper surface. Posts 166 may be cylindrical protrusions which are configured to engage in the holes on the bottom of the first sole. Each post 160 may include a circumferential rib 164 which wraps around the perimeter of post 160. Rib 164 may be located more than half way up post 160 (above post section 166) when measuring from the surface of the sole. The rib may provide a greater engagement with holes 122 in the first sole. The post may also include a groove that extends along the vertical height of the post. This vertical groove may allow for air to escape as the post and rib are pushed the hole. The post may include a rounded top 162 enabling an easier engagement with the holes on the bottom of the first sole. In various embodiments, the plurality of posts 160 may be integrally molded into the second sole. The plurality of posts may be the same material as the second sole. In various embodiments, the posts may be pressed through the bottom of the second sole to extend out of the first sole.

In various embodiments, the second sole includes a tab 158 on the back of the second sole. Tab 158 may extend beyond the surfaces where the first sole and the second sole comes into contact (as shown in FIG. 4). Tab 158 provides a grasping feature to simplify removal of the second sole from the first sole. 152

In various embodiments, the first sole and/or second sole may be manufactured through any process known in the art. For example, the first sole and/or second sole may be manufactured through injection or compression molding. Additionally the support apparatus may be symmetric. For example, any support apparatus may be attached to either a right or a left foot cast.

In various embodiments, the support apparatus as described herein may allow for an improved method of fixing a cast on a patient. As illustrated in FIG. 1a, a patient's foot may be wrapped with a fiberglass, plaster or other casting material (step 210). Apply an adhesive to the first sole (step 220). Apply the sole to the unhardened cast. Place weight in the cast applying pressure to the first sole while the first sole is placed on the floor. While cast is curing the foot is located in a natural and comfortable position (230). The cast and first sole adhesive is then allowed to cure (240). Without the sole in place, the resin of the cast would stick to anything the cast is in contact with, such as the floor. Separating the cast from the floor after curing would damage the cast. Use of the sole prevents this issue by allowing the cast to cure against the sole providing additional bonding to the sole. After the cast resin and the first sole adhesive are cured, the second sole may be applied to or removed from the first sole. When using in conditions where an aggressive tread is preferable (e.g. outdoor use), the second sole may be attached to the first sole. When using in conditions where a moderate tread is preferable (e.g. indoor use) the second sole may be removed.

Systems, methods and computer program products are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to at least one of A, B, and C′ or at least one of A, B, or C′ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed:

1. An apparatus providing support for a cast comprising:
a first sole adhered to the bottom of the cast and
a second sole adhered to the bottom of the first sole.

2. The apparatus of claim 1, wherein the second sole is removably adhered to the first sole.

3. The apparatus of claim 1, wherein the bottom of the first sole includes a surface having a tread pattern.

4. The apparatus of claim 1, wherein the bottom of the first sole includes a plurality of holes suitable for receiving a plurality of studs.

5. The apparatus of claim 1, wherein the second sole includes a plurality of studs on the top of the sole.

6. The apparatus of claim 5, wherein the plurality of studs on the top of the second sole engage the plurality of holes on the bottom of the first sole adhering the two soles together.

7. The apparatus of claim 5, wherein the plurality of studs include a circumferential rib around the stud more than half way up the stud measuring from the top surface of the second sole to the top of the stud.

8. The apparatus of claim 1, wherein the first sole includes a cushion between the upper and lower surfaces of the sole.

9. The apparatus of claim 1, wherein the first sole cushion is thicker at the heel than the rest of the first sole.

10. The apparatus of claim 1, wherein the first sole includes molded trim lines on the top surface enabling simplified alteration to fit a verity of cast sizes.

11. The apparatus of claim 1, wherein the first sole includes grooves on the top surface and side walls which contact the cast, wherein the grooves indicate locations to apply adhesive to improve adhesion to the cast.

12. The apparatus of claim 2, wherein the second sole includes a tread pattern on the bottom of the second sole, wherein the second sole tread pattern is more aggressive than the first sole tread pattern.

13. The apparatus of claim 2, wherein the second sole includes a tab on the back which extends beyond the contacting surfaces of the first sole and the second sole to ease in removal of second sole.

14. A method for applying a cast support comprising:
wrapping a casting material around the lower extremity of a patient;
applying adhesive to a first sole;
compressing an upper surface and sidewalls of the first sole against the bottom and sides of the cast, prior to allowing the casting material to fully cure; and
applying weight in the cast further compressing the first sole and the cast material, prior to allowing the casting material to fully cure.

15. The method of claim 14 further comprising applying a second sole to the bottom of the first sole for outdoor use.

16. The method of claim 14, wherein the adhesive is applied to grooves located on the top surface and side walls of the first sole.

17. The method of claim 4 further comprising cutting the first sole and the second sole along a line molded into the sole to adapt the first sole and the second sole to the size of the cast.

18. The method of claim 15 further comprising removing the second sole from the bottom of the first sole for indoor use.
